(19) United States
(12) Patent Application Publication
(10) Pub. No.: US 2026/0219273 A1
(43) Pub. Date: Jul. 30, 2026

(54) MULTI-LEVEL AND PATIENT-ADAPTIVE BIOMARKER DETECTION METHODS FOR HIGH SENSITIVITY AND SPECIFICITY DISEASE DETECTION AND ASSAYS

(71) Applicant: Orsted Biosciences Corp., Warren, MI (US)

(72) Inventor: Christopher Robert Sedlak, San Jose, CA (US)

(21) Appl. No.: 19/462,665

(22) Filed: Jan. 28, 2026

Related U.S. Application Data
(60) Provisional application No. 63/750,297, filed on Jan. 28, 2025.

(51) Int. Cl. G01N 33/575; G01N 33/543

(52) U.S. Cl. CPC G01N 33/57585; G01N 33/54326; G01N 33/54386

(57) ABSTRACT

The disclosure provides methods of detecting disease correlates using extracellular vesicles, whereby detection of the disease correlates occurs over the surface of the extracellular vesicles without proximity constraints, comprising obtaining a sample of biofluid; using one or more binders to attach to extracellular vesicles correlative to a disease or target organ tissue from the biofluid; measuring biomarkers on the attached extracellular vesicles; upon the measurement not meeting a predetermined detection threshold, disconnecting the binders from the extracellular vesicles; using one or more different binders to attach to the extracellular vesicles; and repeating the disconnecting and the using one or more different binders until the measurement meets a predetermined detection threshold. A sample of those EVs then undergoes approximate single EV analysis in a chamber or capillary.

### RELATED APPLICATIONS

This application claims priority to U.S. Provisional Application No. 63/750,297 filed on Jan. 28, 2025, which is herein incorporated by reference in its entirety.

### BACKGROUND

### Technical Field of the Invention

The technical field of the invention is assays for a disease that has extracellular vesicle disease correlates and including, more particularly, cancer detection. Nevertheless, it could be used for any disease that has extracellular vesicle disease correlates.

### Description of the Related Art

The present disclosure relates to analyzing extracellular vesicles (EVs) for cancer detection, preferably early-stage cancer detection. An extracellular vesicle is a phospholipid bilayer in the shape of a sphere or ellipsoid that gets emitted from a cell. The EV normally contains proteins, fats, carbohydrates, and/or nucleic acids on its surface, within its membrane, and/or inside the EV. EVs are a significant biomolecule holder because almost all their molecules are derived from their cell of origin, EVs have at least 5 times more cancer correlates per milliliter of plasma than cell-free DNA and cell-free RNA in most instances of cancer-positive patients, and EVs contain a relatively large amount of information on average per EV which is highly important to take advantage of the massive benefit that colocalization adds to cancer detection.

Current state-of-the-art early-stage cancer detection generally uses cell-free DNA-based analysis, cell-free RNA-based analysis, or broad protein, DNA, RNA, and fat-based analysis. Circulating tumor DNA (ctDNA) from early-stage cancer is sparse in most biofluids, and DNA typically does not show up unless the cancer cell has already died in most cases. The average subunit is small and contains very little information, making colocalization hard. Cell-free RNA from early-stage cancer is also generally sparse in most biofluids, and RNA is more unstable than DNA. Relative to the average extracellular vesicle, the average unit of cell-free RNA is small and contains very little information, making colocalization hard.

Broad Protein, DNA, RNA, and fat-based analysis has some of the same problems as cell-free RNA and cell-free DNA. High organ-to-organ protein expression overlap leads to false positives and false negatives. High patient-to-patient biomarker variability expression leads to false positives and false negatives. The best existing EV-based analysis currently known uses proximity-based ligation for signal detection. As a result, about 30-70% of the total signal is missed in most instances. This is a largely static approach for all patients with a low ability to adapt to patient-to-patient variability that is innate in humans. If EVs are too close to one another, the proximity ligation-based assays can generate signals from inter-EV ligation events. Thus, in some instances, the specificity of the proximity-based ligation assay is likely reduced by an increase in potential healthy tissue-generated signals.

### SUMMARY DISCLOSURE OF THE INVENTION

A process for cancer detection using extracellular vesicles, includes the steps of obtaining a statistically representative sample of human-derived biofluid; applying Size Exclusion Chromatography (SEC) to the sample to obtain a solution of an enriched sample of extracellular vesicles comprising biomarkers, performing serial release and capture including the following steps (1) attaching a binder to beads and mixing the beads into the solution to bind the binder to biomarkers of extracellular vesicles; (2) placing the beads in a second solution including a binder-to-bead detacher, (3) removing the beads from the solution, and (4) quantifying the number of extracellular vesicles; repeating the serial release and capture steps (1), (2), (3), and (4) with different binders until the number of extracellular vesicles is within a predetermined range, lysing the extracellular vesicles in the solution, mixing binders that correlate with a cancer to the solution comprising the extracellular vesicles, quantifying the number of bound binders that correlate with a cancer in the solution comprising the extracellular vesicles, use a scoring algorithm to determine the likelihood of cancer based on the quantified number of bound binders.

The process may include early-stage cancer detection. The cancer may include lung, liver, nerves, skin, colon, bladder, kidney, brain, intestine, esophagus, pituitary, heart, pancreas, prostate, breast, ovarian, or stomach cancer. The process may include using a protein denaturant. The process may include segregating extracellular vesicles from a solution such that approximately one of the extracellular vesicles enters each of a plurality of chambers.

The disclosure includes methods of extracellular vesicle analysis for preventing cross-contamination of intravesicular contents between neighboring extracellular vesicles comprising segregating extracellular vesicles from a solution such that approximately one of the extracellular vesicles enters each of a plurality of chambers; lysing the segregated extracellular vesicles within said chambers to release intravesicular contents; and analyzing said released intravesicular contents to detect disease-correlative biomarkers.

The disclosure includes methods of detecting disease correlates using extracellular vesicles, whereby detection of the disease correlates occurs over the surface of the extracellular vesicles without proximity constraints, comprising: obtaining a sample of biofluid; using one or more binders to attach to extracellular vesicles correlative to a disease or target organ tissue from the biofluid; measuring biomarkers on the attached extracellular vesicles; upon the measurement not meeting a predetermined detection threshold, disconnecting the binders from the extracellular vesicles; using one or more different binders to attach to the extracellular vesicles; and repeating the disconnecting and the using one or more different binders until the measurement meets a predetermined detection threshold.

Other features and aspects will be apparent from the following detailed description, the drawings, and the claims.

### BRIEF DESCRIPTION OF THE DRAWINGS

FIG. 1 shows an overview of the assay process.

FIG. 2 shows the rest of the overview of the assay.

FIG. 3A and FIG. 3B show a flowchart of the assay process of the disclosure.

FIG. 4 shows an overview schematic illustrating biochemical structures after certain steps and phases of the assay process.

FIG. 5A and FIG. 5B show another flowchart of an overview of the assay process.

FIG. 6 shows an alternative embodiment using microfluidics.

FIG. 7 shows how to obtain approximately one EV per capillary through aspiration

FIG. 8 shows one of the possible ways to lyse approximately one EV already bound to a capillary and a possible way to have binders (each conjugated to a probe) to bind to bind to biomarkers for potential data read out within one capillary or chamber

FIG. 9 shows how to apply a denaturation gradient to a capillary and then obtain signal

FIG. 10 shows a dual-sample method for lung disease detection using both sputum and blood samples.

FIG. 11 is a flow chart that shows an overview of the process of creating the scoring algorithm or scoring equation and the sensitivity and specificity of the assay(s) for each cancer type, disease type, or group of cancers.

FIG. 12 shows Resource-efficient and statistically sound sample sizes based on threshold population size.

The reference numerals refer to the same elements throughout the drawings and the detailed description. The drawings may not be to scale, and the relative size, proportions, and depiction of elements in the drawings may be exaggerated for clarity, illustration, and convenience.

### DETAILED DISCLOSURE OF THE INVENTION

The following detailed description is provided to assist the reader in gaining a comprehensive understanding of the methods, products, and/or systems described herein. However, various changes, modifications, and equivalents of the methods, products, and/or systems described herein will be apparent to an ordinary skilled artisan.

### Modes for Carrying Out the Invention

The present disclosure relates to a patient adaptive iterative feedback-based and gradient-based method for disease detection. Many disease detection assays miss out on coming into contact with crucial disease-causing correlates, and many current approaches do not take advantage of extracellular vesicles. The present invention is extracellular vesicle-centric and greatly increases the amount of disease correlates that are analyzed over other methods. An iterative feedback system enables maximized cancer correlate detection while optimizing patient-to-patient disease analysis.

Advantages of the disclosed multi-level and patient-adaptive biomarker detection methods for improved sensitivity and specificity assays include the assay not requiring two probes to be close to one another to generate a signal. It is extracellular vesicle centric and gets resultantly increased sensitivity and specificity. The method also utilizes patient-adaptive serial capture release of EVs for enhanced sensitivity and specificity and incorporates estimated extracellular vesicle quantity as the assay progresses. The method utilizes and clearly shows how to perform serial capture release. The method incorporates gradient-based protein denaturation for cancer detection. The method is versatile and allows efficient use of resources with iterative particle quantification and potential stop ranges based on the analyzed tissue or organ. The method incorporates amino acid sequence variability finding. The method finds a large amount of disease-correlative biomarkers and places that information into a scoring algorithm or equation.

The user first obtains a sample of biofluid such as but not limited to plasma, sputum, cerebrospinal fluid, and/or saliva. The user then performs size exclusion chromatography on that sample and collects the EV-enriched fractions eluted from the SEC column. The user mixes all or some of these fractions into one tube. The user then takes a fraction of the EV-enriched parent solution, and the user then uses nanoparticle tracking analysis and resistive pulse sensing to obtain the concentrations of particles in the EV-enriched parent solution. (This total particle count can be used later in the algorithm to normalize or adjust the algorithm for patients with highly differing levels of particles in their biofluid). The user then uses a binder-relevant to the condition being tested-bound to a bead, and places it in the EV-enriched parent solution. The binder beads are then mixed for binding to specific biomarkers in the sample. The beads are then washed once the binder is given enough time to bind to its biomarkers. The washing step removes a large number of unbound biomolecules in the solution. An enzyme or another chemical reagent is then placed in the parent solution to cause the binders to detach from their beads. These detachers would ultimately depend on the setup. For instance, if a PEG-biotin linker is used to attach a binder to beads, then biotinidase can be used to break the binder's link to the bead. The beads are then removed from the solution. A fraction of the resultant solution is then taken aside for quantification. The serial capture release step is repeated until the estimated particles or EVs fall within a desired range.

Then, the user repeats the previous process but with a probe that can be measured, such as a fluorophore, since the user might need more correlates for the assay. The user proceeds to wash the beads and proceed to the Capillary phase. The binders are detached from the beads using an enzyme and quantified with NTA and resistive pulse sensing. Using that information and concentration, the user dilutes the resultant liquid such that a representative sample size of EVs (based on Table 6 and the graph below) go into approximately one EV per capillary. The user chooses to use capillaries pre-coated with Protein G. The user lyses the EVs with EV lysing reagents. Then, more protein surface area originating from the EVs have more binding site exposure. The user uses antibodies to bind the biomarkers and keep them indirectly bound to the capillary walls. The resultant capillaries are washed to remove unbound material. Then, the user partially (or fully) denatures the proteins and adds affimer-bound-probes to the capillaries. Then, the user takes that data and all other sets of data throughout the process to determine if the individual has a condition and the confidence or probability of having that condition. Thus, users can find an abundance of disease correlates and place them into a scoring algorithm or equation specific to the disease/condition they are looking for.

In instances where EVs are collected within a mesh and dried and then resuspended in a solution, SEC may not be needed for EV processing. In some instances, the bead could have two or more unique binders bound to the beads to capture multiple unique biomarkers. The probe addition step within (20) at the end of the serial capture release phase may be skipped and achieve similar results in some instances.

The serial capture release phase can have a number of setups with many different binders and a variety of other linkers and enzymes. One example is using IdeZ proteases to cleave antibody hinges off antibodies to detach bound EVs and particles from beads. Biotinidase can be used to release biotin and detach biotin-linked antibodies from beads. Capillaries can be replaced with any setup that enables approximate single EV analysis, subsequent amino-acid sequence analysis, and further protein analysis.

Some of the serial capture release phase cycles may be bypassed with a microfluidics setup that reads the number of probes bound to an EV or biomolecule aggregate and separates them into separate chambers based on the number of unique probes or the total number of probes bound to an EV or biomolecule aggregate. A microfluidics device may also be used to perform the particle quantification steps.

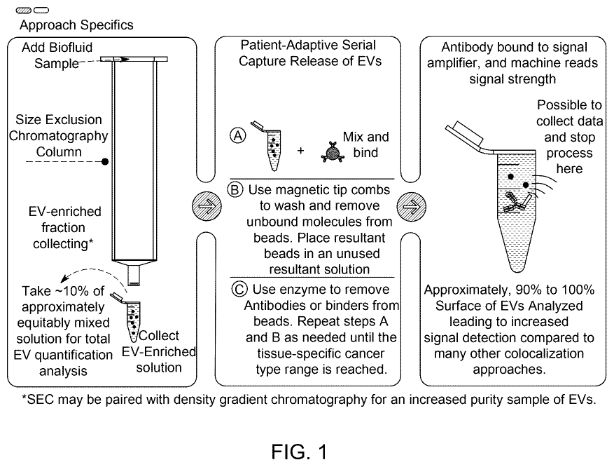

FIG. 1 shows a partial overview of the disclosed process. The process starts with extracellular vesicle isolation. Size exclusion chromatography (SEC) is presently one of the best singular methods for EV isolation. It keeps the EVs intact while removing much of the superfluous non-cell origin biomolecules from the surface of the extracellular vesicles. Serial capture release of EVs using antibodies specific to cancer-predictive biomarkers is performed. Then, read off the signal. The serial capture release phase alone gives approximately 2-10 times the information as the next best EV-based cancer detection test.

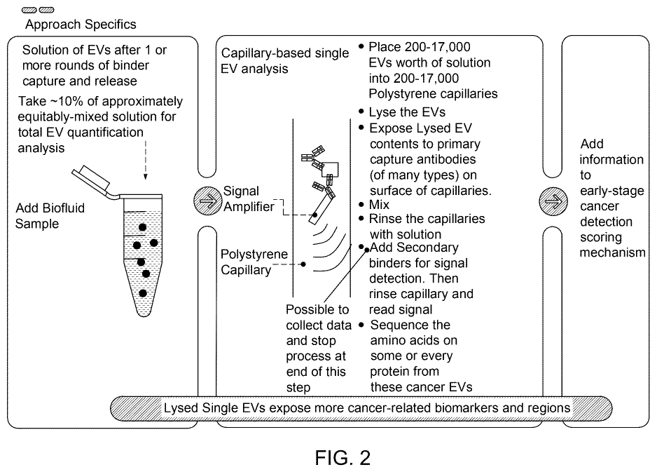

FIG. 2 shows the rest of the overview of the disclosed process as a continuation of FIG. 1. This method can include separating and lysing EVs in an extensive capillary system—in which further proteomic analysis and protein sequencing are done to find additional amino acid sequences and biomarkers that correlate with cancer. The problem to be solved is that current state-of-the-art assays have a poor ability to detect disease correlates, and their inability to detect a large fraction of these correlates frequently leads to a high rate of false positives and false negatives, especially for many early-stage cancers. The high level of inaccuracy causes these assays not to be applicable to the general population because—for many of these tests—if they were applied to the general population, approximately 90% of the people who test positive for the disease do not have the disease in some instances (especially with lung cancer assays). This problem usually stems from patient-to-patient variability, wide proteome expression variability among healthy patients, analyzing biomolecule types that are sparse and have low colocalizationability, methods that do not mitigate high background noise that stems from high background solutions such as plasma, and static noniterative and non-optimizing assay processes. The disclosed methods overcome this problem by analyzing as many disease correlates as possible. The mid-assay particle quantification while loop (or adaptive section) largely ensures that a serial capture release phase does not occur too much or too little such that the end signal output is not catastrophically riddled with data generated from biomarkers that are from nontarget tissues and organs (from stopping serial capture release phase above the listed EV ranges) while ensuring that we confidently and efficiently identified enough disease-causing correlates (by not going below the specified stop ranges) to predict—with high accuracy—whether or not an individual has the disease.

The methods lay out a largely maximized extracellular vesicle surface area analysis (by effectively analyzing close to 90% to 100% of the EV's surface area and largely not making signal output contingent upon a proximity-based constraint). The assay then enables significantly increased biomolecule surface area analysis by lysing EVs, thus drastically reducing lipid membrane-based steric hindrance from the region the binder is specific to. Further disease correlate analysis is done with gradient-based protein denaturation and amino acid sequencing analysis. Based on a Central Dogma of Molecular Biology, a large fraction of cancer-related correlates should be found in amino acid sequence deviations.

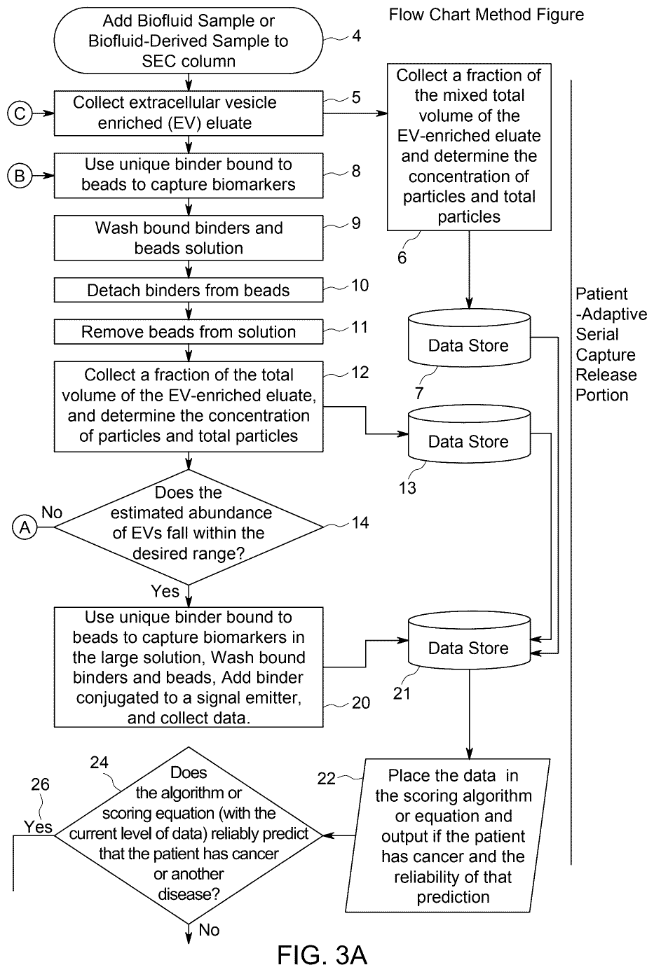

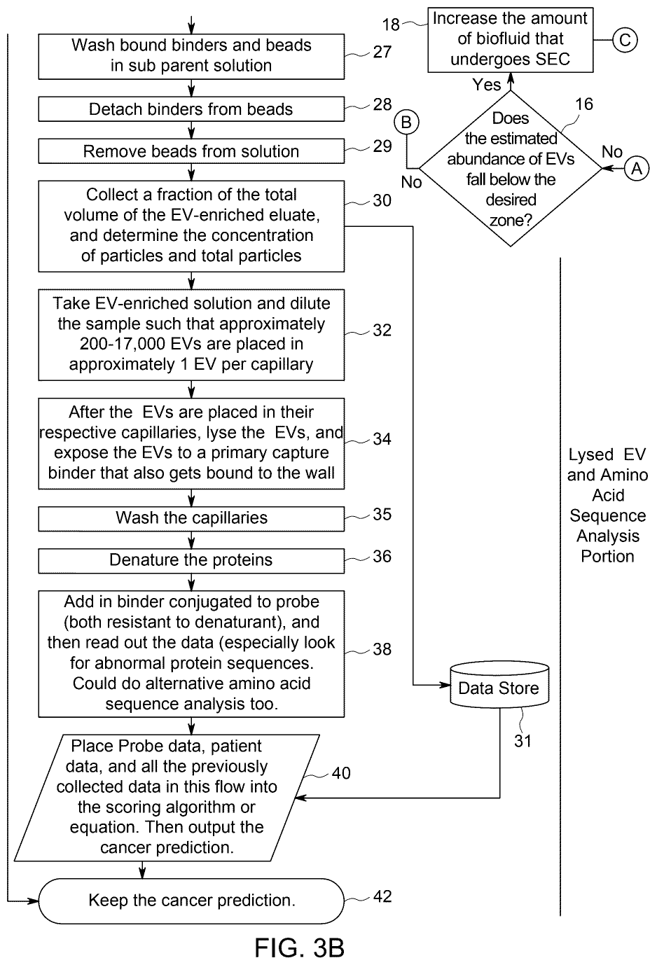

FIG. 3A and FIG. 3B show a flowchart showing an overview of the assay process. The process is separated into two main parts: a serial capture release of extracellular vesicles part and a lysed EV analysis portion.

4 is the addition of a biofluid sample or biofluid-derived sample to a Size Exclusion Chromatography Column. Fractions of SEC eluate 5 are then collected. A subset of the EV-enriched eluate is removed from the main workflow and taken aside for particle quantification at 6. 7 is data storage for the estimated total quantity of particles in fraction of SEC to be analyzed. 8 adds binders bound to beads to bind to specific biomarkers. 9 washes the parent solution to remove unbound molecules. 10 detaches binders bound to the beads in the parent solution. 11 removes beads from the parent solution. 12 collects a fraction of the parent solution, quantifies the total, and generates an estimated amount of bound EVs, molecules, and protein aggregates. 13 stores the estimated particle concentration and total particle data for later processing. 14 is a decision that is made based on the estimated abundance of EVs falling within a certain range. If the EV or particle estimate does not fall within the estimated abundance of EVs range, then the process proceeds to the “no” pathway to 16. 16 is a decision that is made for determining if the EV estimate falls below the desired range. If the estimate is above the desired particle range, then the process proceeds to the no pathway starting at 8. Otherwise, the process proceeds to the yes pathway to 18.

18 increases the total amount of analyzed sample and processes the sample through SEC and then proceeds to 5. If the estimated abundance of EVs at 14 falls within the desired range, the process proceeds to the yes pathway to 20. 20 is a process that captures biomarkers with beads, cleans the beads to remove unbound biomarkers, adds a biomarker bound to a probe, and then data is collected. That data then proceeds to a data store 21. Data store 21 collects all the data from the previous data stores 7 and 13. Data store 21 places that data in 22. 22 is a scoring algorithm that marks the end of the serial capture release portion of the assay. 22 determines if someone has cancer and its level of certainty. The scoring information and level of certainty goes to 24. If the reliability of the prediction is above a certain threshold, the process proceeds to the “Yes” pathway 26 to keep the cancer prediction result 42. Otherwise, the process proceeds to 27 shown in FIG. 3B

The line on the left side of FIG. 3B is the Yes-26 line shown at the bottom left in FIG. 3A. The top of 27 is shown at the bottom of FIG. 3A and at the top of FIG. 3B. Thus, FIG. 3B is a continuation of FIG. 3A.

27 is the process of washing the bound binders and beads to potentially remove unbound material. That proceeds to process 28. That parent solution proceeds to 28. 28 is the process of detaching binders from beads in the parent solution. The unbound beads are then removed from the parent solution in process 29. A fraction of the resultant parent solution is removed from the process for particle quantification and EV concentration estimates in process 30. That information is then stored in 31. A subset of the parent solution is diluted into 200-17,000 EV capillaries for approximate individual EV analysis in process 32. The solutions in capillaries then get a solution containing EV-lysing solution and primary capture binders—that also get bound to the capillary walls—added to the capillaries in process 34. The capillaries are then washed in process 35. The bound material is then exposed to conditions that cause proteins to denature in process 36. The capillary solution then gets a solution containing probe-bound binders added to it, and then the probe data is read out in process 38. The final probes data and all particle quantification data are then placed in a scoring algorithm or equation in 40. 40 then outputs the cancer prediction. The prediction and reliability of the prediction are then kept in the end step of 42.

Capillaries getting coated in protein G is also a possibility. It would just have to be ensured the capillary walls remain reasonably transparent. One could also use math and empiricism to distinguish between the EVs and lingering binders in the solution. The EV quantification data could be used to reduce binder-bead usage as the assay progresses. It would also be fine to avoid protein denaturation.

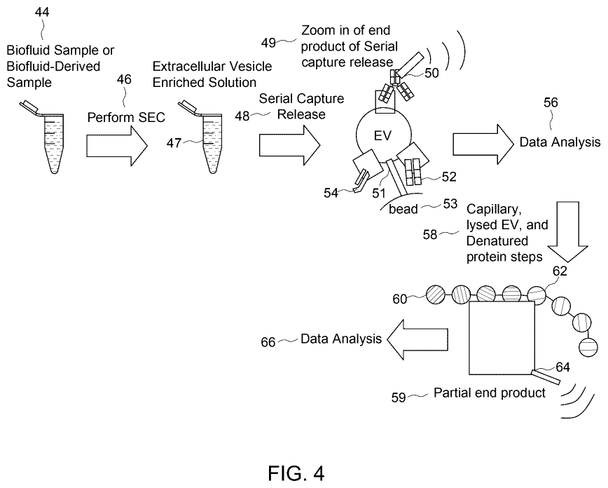

FIG. 4 is an overview schematic illustrating the core biochemical structures after key steps and phases from the Flow Chart shown in FIG. 3A and FIG. 3B. The process starts with a biofluid or a biofluid-derived sample in 44. That sample then undergoes size exclusion chromatography in 46. Some fractions of the SEC eluate are then collected in 47. 47 is a collection of some fraction of SEC. That fraction of SEC contains EV-enriched eluate. The EV-enriched eluate then undergoes a serial capture release phase 48. 49 is the zoomed-in end product of the serial capture release. It shows an extracellular vesicle with three biomarkers on its surface. Each of those biomarkers has a bound binder on them. 50 is an antibody with a probe attached to it. 52 and 54 are binders bound to the biomarker, but their original attachments to beads were removed, potentially through a binder or linker breakdown. 53 is a bead that is bound to a binder 51. 51 is a binder that is bound to an EV. The probe information and other information gathered during the serial capture release phase are placed into an algorithm 56. If the algorithm needs more information to give a more reliable result, the parent solution gets further processing in the capillary, lysed EVs with lysing solution, and denatured protein steps 58. 59 shows a zoomed-in partial end product after the second phase is complete. 60 is a denatured protein. 62 is part of 60 and is a portion of the amino acid sequence that binder 64 is specific to. 64 also has a probe attached to it to enable signal detection in the capillary. That data and all the previous data generated in the assay then go into a scoring equation or algorithm 66 specific to the cancer or disease condition to generate a score and disease prediction.

SEC may not need to be performed for samples with a low concentration of EVs and in biofluids with low background signals generated from off-target organs and tissues. Compared to some proximity ligation assays, the serial capture release phase alone will get you approximately 2-10 times more data by analyzing almost the total surface area because there is no proximity constraint for signal to be picked up by a reader. The capillary analysis phase further adds to the total amount of cancer correlates you can find. Placing roughly 1-2 EVs in a capillary and then lysing the EVs exposes more cancer correlates that would have been blocked from steric hindrance. Then, you expose even more cancer correlates in the amino acid sequences and level of protein structures by denaturing the proteins. Adjusting levels of protein denaturant theoretically would correlate with cancer, so having a gradient of denaturing solutions should correlate with cancer-correlative biomarkers. Also, tailoring denaturation to get to specific proteins' secondary or primary structure can help get more cancer correlates for specific disease detection purposes. It is also important that EVs can be lysed and have probes added to the capillary solution with or without protein denaturation taking place.

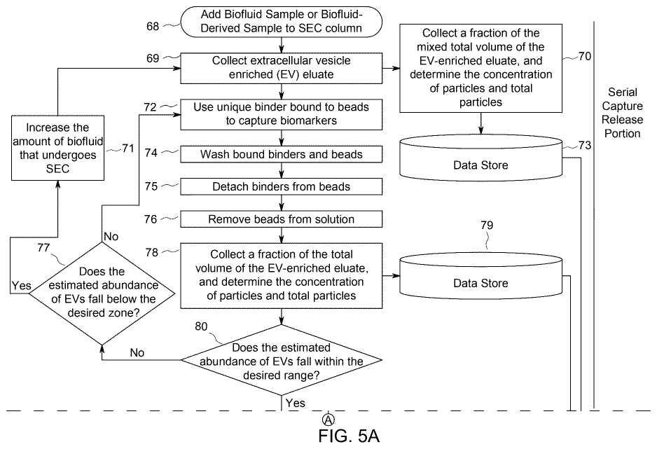

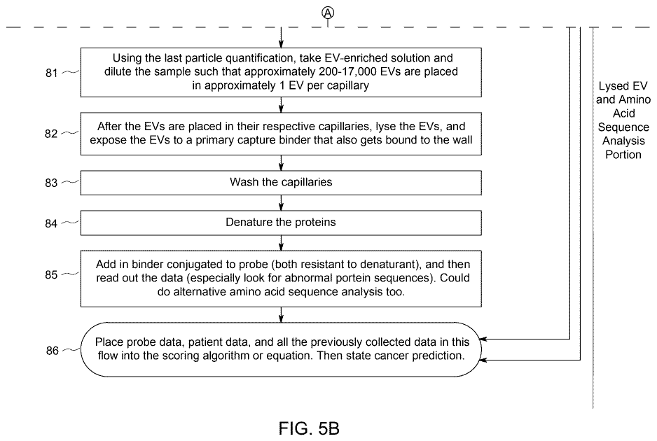

FIG. 5A and FIG. 5B together show another flowchart showing an overview of the assay process.

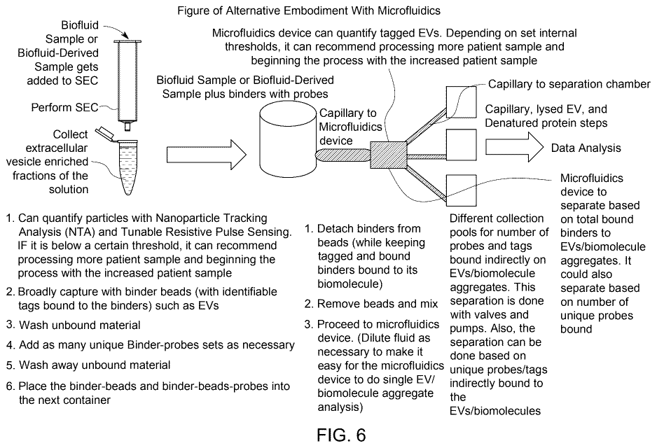

FIG. 6 shows an alternative embodiment using microfluidics. In some instances, SEC may not need to be performed for samples that have a low concentration of EVs. In some instances, serial capture release cycles can be removed with a microfluidics-based approach.

In some instances, proteins do not need to be denatured in the capillaries. However, in many instances the increased biomarker exposure from denaturation will still lead to increased cancer correlate exposure. In some instances, you can skip to the probe step and place it in the algorithm at the end. The last step does not have to be denatured protein analysis. The last set of binder probes could bind to any biomolecule type (ideally correlative to a disease).

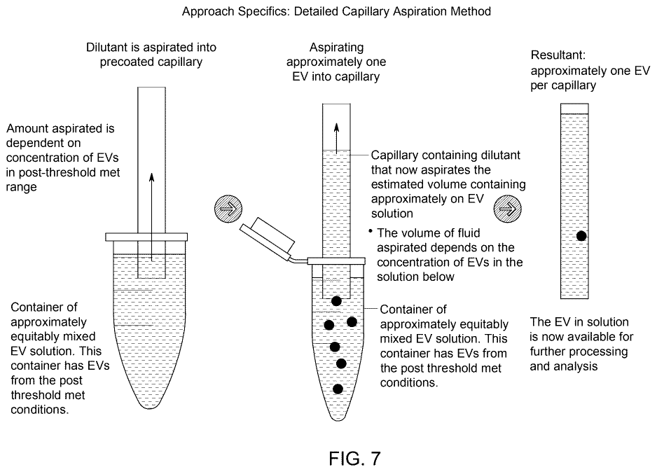

FIG. 7 shows the details of isolating approximately 1 EV per capillary. FIG. 7 provides clarification on how an approximate single-EV per chamber is obtained, which is typically performed after the EV threshold boundary condition is completed. A precoated capillary aspirates a dilutant solution into the capillary. The dilutant volume is dependent on the estimated concentration of EVs in the resultant threshold-met parent solution. Likewise, the amount of aspirated threshold-met parent solution would be the volume most likely to yield one EV. Specifically, it would be the amount of volume that is expected to contain approximately one EV (based on an earlier EV concentration analysis). The capillary ends up with roughly one EV. Approximately one EV per capillary enables further processing and analysis for later steps in the listed methods.

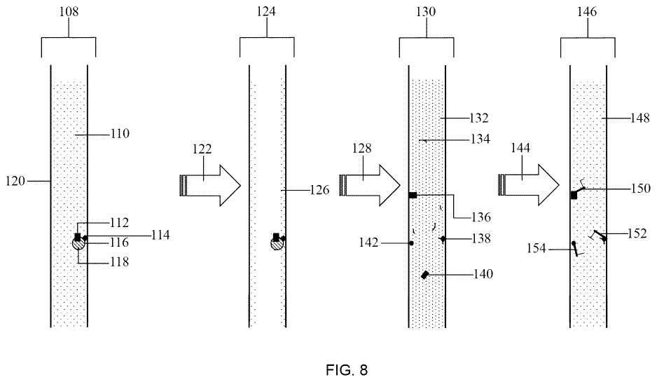

FIG. 8 shows one of the possible ways to lyse approximately one EV already bound to a capillary and a possible way to have binders (each conjugated to a probe) to bind to bind to biomarkers for potential data read out. 108 shows a section of a capillary in which its walls (120) are interiorly pre bound with a diverse population of lysing-solution resistant binders and blockers. 116 is immersed in 110. In 108, is one EV (116) in which one of its surface molecules (114) are covalently bound to one of the binders on the capillary walls. 118 is the interior of the EV which contains its cargo. 112 is its transmembrane protein. In 122, the majority of the 110 gets ejected. 124 shows the resultant. 126 shows that there is residual isotonic solution left behind. This occurs for a short period of time. In 128, lysing solution gets added to the capillary. 130 show the resultant of adding lysing solution (132). In that lysing solution, are broken apart EV membrane fragments (134), and an unbound protein (140) that was released from the EV. 142 is a carbohydrate that is now bound to one of the binders on the capillary wall. The transmembrane proteins interior portion (which was correlative for a disease) (136) now binds to a binder on the wall of the capillary. 138 is 114 but without the majority of the EV attached to the biomolecule. 144 is a four-part process. The first is a washing process that removes unbound EV membrane fragments, unbound proteins, and other EV cargo. The second process is adding a variety of binders bound to probes to the capillary. The third process is washing away unbound binders bound to probes. The fourth process is aspirating isotonic solution into the capillary. 146 shows the resultant. 148 is the isotonic solution. 150, 152, and 154 are probes conjugated to distinct probes. 150, 152, and 154 are all bound to their biomarkers.

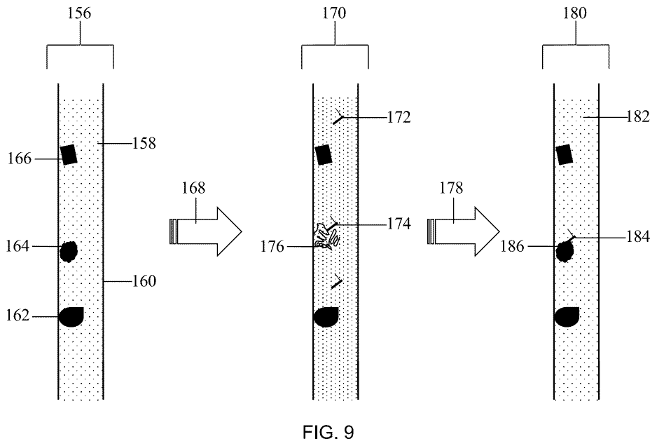

FIG. 9 shows a possible denaturation gradient process that can be applied to a capillary to check for additional cancer-specific biomarkers. Specifically, it is a heat-based partial denaturation process applied to the solution in the capillary. The process starts with a capillary containing isotonic solution (158) and three proteins (162, 164, and 166)—from lysing an EV and washing away molecules not directly or indirectly bound to the interior walls of the capillary. The internal capillary walls (160) are coated with denaturation resistant binders covalently bonded to the capillary and denaturation resistant blockers. 168 is a two-step process. The first step is to dispense the isotonic solution and aspirate a solution containing heat-resistant binders conjugated to probes. The second step is to slowly heat that solution from approximately 25° C. to 50° C. and give time for the binders to bind to any specific structures that get exposed. This results in what is shown in 170. 166 an 162 remain in their native states. However, 176 shows partly denatured protein that now has 174 bound to the internal secondary structure on Protein 2b. There are also unbound binders such as 172. 178 is a two-step process. The first step is slowly allowing the solution to slowly cool to room temperature. The second step is washing the unbound probes from the solution and then aspirating isotonic solution into the capillary. The resultant is shown in 180. There is one binder (184) bound to 186. The data can then be read off from information released from the probe. Sometimes DNA mutations—within a cell—related to cancer cause downstream amino acid changes in proteins that can cause the protein to be less resistant to denaturing conditions in general. In many instances, these atypically denaturable proteins can serve as good indicators for cancer if identified.

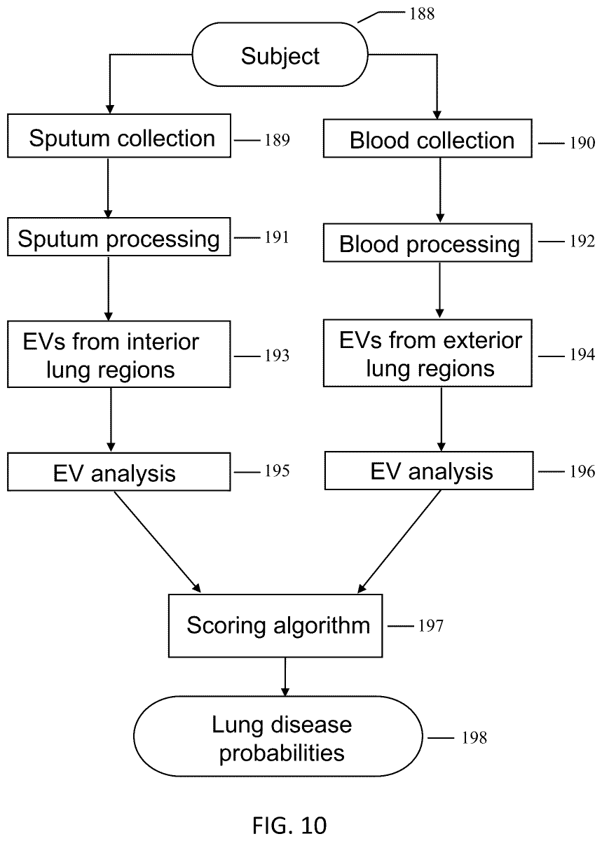

FIG. 10 shows a dual-sample method for lung disease detection. In some aspects, lung disease detection may be performed using both a sputum sample and a blood sample obtained from the same subject 188. Sputum contains extracellular vesicles that originate primarily from cells lining the interior airway surfaces of the lungs, including the bronchi, bronchioles, and alveoli. Blood, such as plasma or serum, contains extracellular vesicles that include those originating from cells on the exterior surfaces of lung tissue that interface with the pulmonary vasculature and surrounding structures. Because lung diseases, including early-stage lung cancer, may arise in either interior airway-exposed regions or exterior vascular-exposed regions, analyzing extracellular vesicles from only one biofluid source may fail to detect disease correlates present in the other region. Additionally, extracellular vesicles from certain lung regions may be more readily captured in sputum than others. For example, extracellular vesicles originating from larger airways such as the bronchi may be more easily expectorated and collected in sputum, while extracellular vesicles from smaller peripheral structures such as distal bronchioles or alveoli may be less likely. In such instances, blood-derived extracellular vesicles may provide complementary detection of disease arising in these harder-to-reach regions.

Referring to FIG. 10, in the sputum path, sputum is collected at 188, processed at 190, yielding EVs from interior lung regions at 191, which undergo EV analysis at 193. In the blood path, blood is collected at 190, processed at 192, yielding EVs from exterior lung regions at 194, which undergo EV analysis at 196. By obtaining and analyzing both sputum and blood samples from a subject, the method may detect extracellular vesicles shed from a broader range of lung tissue, thereby increasing sensitivity for lung disease detection. In some instances, the extracellular vesicle analysis performed on each sample may comprise segregating extracellular vesicles such that approximately one extracellular vesicle enters each of a plurality of chambers, lysing the segregated extracellular vesicles within the chambers to release intravesicular contents, and analyzing the released intravesicular contents to detect disease-correlative biomarkers. In some aspects, proteins within the released intravesicular contents may be denatured to expose amino acid sequences correlative to lung cancer.

Results from both the sputum path and the blood path are combined in scoring algorithm 197 to determine lung disease likelihood at 198. In some instances, the scoring algorithm may weight biomarkers from each sample type differently based on their correlation with specific lung disease subtypes or stages. For example, certain biomarkers found predominantly in sputum-derived extracellular vesicles may correlate more strongly with diseases affecting central airways, while biomarkers found predominantly in blood-derived extracellular vesicles may correlate more strongly with diseases affecting peripheral lung tissue or the lung parenchyma. By combining data from both sources, the method may achieve increased sensitivity and specificity compared to single-sample approaches, particularly for early-stage lung cancer detection where disease may be localized to a specific lung region.

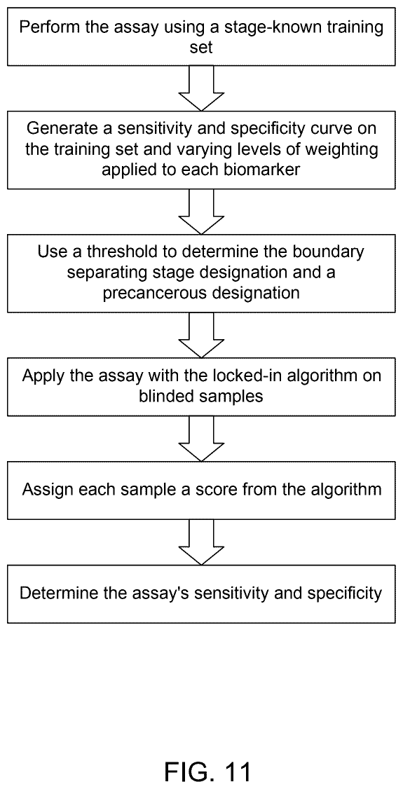

FIG. 11 shows an embodiment of a scoring algorithm of an assay of the disclosure.

### Glossary

Size exclusion chromatography (SEC) is a technique that separates molecules based on their size. It works like a molecular sieve, allowing larger molecules to pass through first while smaller ones take longer, helping scientists analyze and purify different substances effectively.

Serial capture-release is a method that isolates specific molecules by using special binding agents that “catch” them. This technique allows the repeated capture and release of these molecules, which makes studying proteins or other vital substances in complex mixtures easier. The disclosed method uses patient-adaptive serial capture release with range-based EV quantification.

The disclosed Patient Adaptive (or EV Quantity Threshold) Serial Capture Release is a novel early-stage cancer detection method.

The threshold for serial capture release is a high-utility release method that accounts for patient-to-patient variability in EV quantity, gene expression, and protein expression while minimizing resource waste. Each serial capture release cycle whittles down the EV population to be progressively organ-specific and cancer-specific until it reaches a specific range based on the biofluid analyzed, the volume of biofluid, and the organ type to be investigated.

Novel Single Extracellular Vesicle Analysis Done Across (200-17,000) Capillaries presents a novel cancer detection method. The capillaries enable approximate single extracellular vesicle analysis. Each capillary receives approximately one EV per capillary. The separate capillaries enable many EV analysis techniques, such as lysing EVs to expose more cancer correlate sites, subsequent protein denaturation steps for amino acid sequence-based cancer correlate finding, and finally, binder-probes-based signal readout.

The number of capillaries used depends on the estimated quantity of organ or tissue-derived EVs that fell within the stop threshold range. The number of single EVs to analyze is based on the minimum needed EVs to have a sample set representative of the population of EVs being analyzed. Namely, once the estimated amount of organ or tissue-derived EVs is found within the threshold, the number is chosen as the minimum sample size with a confidence level of roughly 99% and a margin of error of approximately 1%. This process ensures that excess reagents, binders, and capillaries are not used while maintaining a statistically representative sample size of the previous population.

As used herein, “approximately one extracellular vesicle” or “approximately one EV” refers to a distribution of extracellular vesicles into chambers such that chambers containing extracellular vesicles contain, on average, a single extracellular vesicle. This may be achieved through methods including, but not limited to, dilution, segregation, statistical distribution, active sorting, or detection-based selection. Chambers that do not receive an extracellular vesicle may be excluded from subsequent analysis.

As used herein, “capillary” refers to any elongated chamber, channel, tube, or compartment capable of receiving and containing a solution comprising extracellular vesicles for analysis. This includes but is not limited to glass capillaries, polymer capillaries, microfluidic channels, and other elongated compartments.

As used herein, “quantifier” refers to any device or method capable of measuring or estimating the quantity or concentration of extracellular vesicles in a solution. This includes but is not limited to nanoparticle tracking analysis devices, resistive pulse sensing devices, flow cytometers, optical detection systems, or other particle counting methods.

As used herein, “scoring algorithm” refers to any computational method for determining disease likelihood based on detected biomarkers. This includes but is not limited to weighted sums, logistic regression, support vector machines, random forests, neural networks, or other machine learning classifiers.

As used herein, “lysing” refers to disrupting extracellular vesicle membranes to release intravesicular contents for analysis. Lysing may be achieved through methods including but not limited to chemical reagents, detergents, osmotic shock, sonication, or other membrane disruption techniques

### Example 1

### Lung Cancer Test

A biofluid sample is collected from a patient and then processed in a lab using a combination of laboratory extracellular vesicle separation/isolation and analysis techniques.

Step 1: A 1 ml sample of human-derived biofluid, such as blood plasma, is taken from a patient.

The process of Size Exclusion Chromatography (SEC) begins.

Step 2: The 1 ml sample undergoes size exclusion chromatography to obtain an enriched sample of extracellular vesicles (EVs). This results in the EV-enriched size exclusion eluate, which contains 5 billion EVs.

The process of patient-adaptive serial capture release begins.

Step 3: Binder A, attached to a ferromagnetic bead, is then allowed to mix in the solution to bind to biomarkers.

Step 4: The EV-bound beads are placed in a new solution that contains binder-to-bead detachers and then mixed.

Step 5: The magnetic beads are then removed from the solution (such as with magnetic tip combs closely covered in plastic to prevent the beads from directly touching the magnet).

Step 6: The solution is mixed (to distribute the particles equitably).

Step 7: Then, a fraction of the EVs are quantified using two methods to ensure accuracy (tunable resistive pulse sensing and nanoparticle tracking analysis). Based on that quantification, the estimated number of EVs is 90 million.

Step 8: The previous capture release steps (Steps 3-7 above) are repeated, but with Binder B replacing Binder A. The estimated number of EVs is now 8 million.

Step 9: The previous capture release steps (Steps 3-7) are repeated with Binder C replacing Binder B. The estimated number of EVs is now 80 thousand. (The process theoretically repeats until a range of 59,200-1,184,000 EVs is estimated to be in the parent solution; the process can stop outside of 80 thousand EVs).

EV capillary analysis process begins.

Step 10: The previous solution is then mixed to distribute EVs evenly within the solution. The capillary aspirates a set amount of fluid to obtain roughly one EV in its capillary. The aspiration process is done for 13,739 capillaries. Then, a set amount of EV-lysing solution is added to each capillary in the 13,739 EV capillary system. Each EV membrane breaks apart and exposes additional binding sites on the proteins. (Each EV is effectively broken apart completely).

Step 11: Primary binders are added to each capillary to bind to the proteins and other biomarkers that correlate with cancer. The primary binders also bind to the capillary walls.

Step 12: A washing solution is then added to the capillaries to remove all material that is not directly or indirectly bound to the capillary wall.

Step 13: Protein denaturing solution is added to each capillary.

Step 14: A flurry of binders is added to each capillary to check for disease-correlative (either positively or negatively correlated) amino acid sequences. The specific combination is adapted for each target biomarker. (Note: these binders are not designed to bind to the capillary wall like the previous binder set.)

Step 15: Once the binders have enough time to bind, the capillaries are washed to remove unbound binders and protein denaturant. (However, the washing solution maintains the denaturing effect. The binder probes are denaturation resistant and used in the next step).

Step 16: Then, the probes get exposed to a stimulant (such as visible light or another specific range of electromagnetic frequency) that causes the bound probes to emit a detectable signal (unique to each binder type).

Step 17: A reader reads out the signals from each capillary. (The reader is a sensor that can detect the electromagnetic radiation ranges and intensity emitted from the probes.) During that process, the reader distinguishes between each probe type in the information collection process.

Step 18: The probe data picked up from the reader, and EV quantity data are then added to the scoring algorithm.

The patient scored a 12 on our scoring algorithm for lung cancer, so we conclude with a high level of confidence that the patient has stage 2 lung cancer because the patient exceeded our threshold score of 8.

Extracellular vesicles are lipid spheroid and ellipsoids that bleb off from cells. These lipid spheres have proteins, carbohydrates, and other biomolecules on and within them that are unique to the cell they originated from. The information these EVs contain is unique to their cell of origin, plus their abundance in biofluid makes them the ideal biomolecule holder for detecting early-stage cancer.

Binder A is a generic binder. This binder could be specific to antigens that are specific to the organ type you're analyzing or a cancer correlate. These binders can be antibodies, affimers, or other subtypes of binders. The capture binders will likely be organ-specific early in the patient-adaptive serial capture release process. In contrast, the later binders are likely going to be cancer-specific correlates.

A binder-to-bead detacher is a solvent, enzyme, or electromagnetic radiation that is used to break a bond or bonds that keep the binder wholly attached to the bead. Ultimately, the binder-to-bead detacher aims to remove the indirectly bound extracellular vesicle from the bead.

This step allows for a dramatic increase in surface area for the remaining steps. Removing these large beads removes a significant obstacle to binding in later steps. With each additional binder increasing the reliability of the test.

The idea of approximate single-EV analysis within capillaries is novel and allows for significant test sensitivity and specificity increases. It is also essential to provide a chamber to apply powerful analysis methods (including lysis to remove steric hindrance for some disease correlates, protein denaturation, and adding new binders for determining amino acid sequence) with drastically reduced contamination from other EVs or cells that could generate false positives in your assay.

In some embodiments, lysing serves as a step in extracellular vesicle (EV) analysis by disrupting EV membranes to release their internal contents for further examination. This can provide access to the intravesicular content, facilitating molecular assays and cargo analysis . . .

Once the estimated number of EVs falls within the target range (59,200 and 1,184,000 per mL processed), a fraction of the medium is placed in a 200-17,000 capillary system such that approximately one extracellular vesicle ends up in each capillary. The EV target range is a novel idea in extracellular vesicle analysis because it ensures that you are reducing off-target tissue and organ analysis while at the same time maximizing the ability to detect correlates in the target tissues and organs.

In this example, a primary binder is either prebound to the capillary or a binder that ultimately gets bound to the capillary. This binder is necessary for keeping desirable biomolecules in the capillary during the washing steps.

Denaturation is important because cancer-correlative DNA mutations—that may not cause proteins to be dysfunctional—can oftentimes alter the proteins' primary sequence. So, denaturation (which causes proteins to unfold) exposes these abnormal sequences that can correlate with cancer. The protein does not necessarily need to be fully denatured to its primary structure to find these cancer correlates; the protein can be denatured down to its secondary structure to find these correlates.

A probe is a term used to describe a molecule that gives off a specific signal once excited by an outside source such as electromagnetic radiation. For example, a probe may emit red light once it absorbs violet light from its environment.

The scoring algorithm was previously determined by running the assay using a stage-known training set. Then, once a sensitivity and specificity curve was generated on that training set, varying levels of weighting were applied to each biomarker for the algorithm. A threshold of 8 was then used to determine the boundary separating stage 1 designation and a precancerous designation. We then applied the assay with the locked-in algorithm on a very large number of blinded samples. Each sample received a score from our algorithm. Then, based on that large-scale experiment, we determined our assay's actual sensitivity and specificity.

### Example 2

This Example shows three main types of high utility ranges. The first type contains the minimum recommended volume of biofluid to begin the process based on the organ or tissue type to be analyzed. This range was derived from Cochran's sampling equation and from minimizing the effect of loss from size exclusion chromatography. Specifically, the ranges listed in the tables below (specifically Table 1 and Table 2) should significantly reduce sample variability that would occur from normal variation within an EV population.

The second type of potentially patentable ranges contains the optimal thresholds to discontinue the serial capture release phase based on the volume of biofluid initially processed in the assay. These thresholds will yield approximately the best sensitivity and specificity in a cancer assay. Specifically, these thresholds should represent the population of EVs unique to the target organ and cancer type. All these thresholds for category two are in Table 3, Table 4, and Table 5.

The third category is for sampling ranges once the post-serial capture release threshold is met. Specifically, this is the number of EVs undergoing single-EV analysis within the capillary phase. These sample sizes are for efficient resource use and obtaining a sample size that you are roughly 99% sure represents the EV population at the end of the serial capture release phase. Some ideal sampling sizes are listed in Table 6. The gaps are filled in by extrapolating from FIG. 1.

Type 1: Minimum Volume Needed to Start Off in the Method for Low (Not Significant) Variability For each Biofluid Type and Tissue Type.

TABLE 1
Approximate Minimum Starting Volumes for the Method for
Analyzing Specific Tissue Types Within Plasma. Minimum
Processed Starting Volume of Plasma Needed for Low-Variability
Analysis of Tissue-Derived EV Populations.

| Tissue-Derived EV Type | Minimum Volume (mL) |
|---|---|
| Adipose Tissue | 0.002032 |
| Muscle | 0.02728 |
| Lung | 0.05622 |
| Liver | 0.09194 |
| Nerve | 0.10599 |
| Skin | 0.13529 |
| Colon | 0.14727 |
| Bladder | 0.19128 |
| Kidney | 0.2280 |
| Brain | 0.2600 |
| Small Intestine | 0.5547 |
| Esophagus | 0.6656 |
| Pituitary | 0.6656 |
| Heart | 0.7563 |
| Pancreas | 4.1575 |
| Stomach | 8.309 |

This table shows the approximate minimum starting volume you need for low variability analysis of specific organ and tissue types within plasma. This table assumes that the plasma sample was taken from a parent solution that was approximately equitably mixed from a starting volume of roughly 0.25 mL or higher to match what is shown in the table.

TABLE 2
Minimum Starting Volume for the Method for Analyzing
Lung Tissue Within Sputum. Minimum Processed Starting
Volume of Sputum Needed for Low-Variability Analysis
of Tissue-Derived EV Populations

| Tissue-Derived EV Type | Minimum Volume (mL) |
|---|---|
| Lung | 0.0066344 |

This table shows the approximate minimum starting volume you need for low variability analysis of lung tissue within sputum. This table assumes that the sputum sample was taken from a parent solution that was approximately equitably mixed from a starting volume of roughly 0.25 mL or higher.

### Type 2: Serial Capture Release Stop Thresholds

TABLE 3
Serial Capture Release Stop Thresholds for Analyzing Hematopoietic-
Derived EVs. Estimated EV Threshold Range per mL of Plasma
Processed for Hematopoietic-Derived EV Types

| | Lower Range | Upper Range |
|---|---|---|
| Hematopoietic-Derived EV Type | (EVs/mL) | (EVs/mL) |
| Neutrophil | 1297400 | 25948000 |
| Red Blood Cells | 3972040 | 79440800 |
| B Cells | 25708480 | 514169600 |
| Monocyte | 3453080 | 69061600 |
| CD4 T cell | 11417120 | 228342400 |
| CD8 T cell | 3063860 | 61277200 |
| Platelet | 50898000 | 1017960000 |

This table shows the stop thresholds for analyzing EVs in an assay for cancers related to these EV types. Note that this table is for the volume of plasma initially inputted into the process. For example, if you initially inputted one milliliter of plasma into this process, your lower stop threshold range would be 25708480 EVs for analyzing the B cell EV population. Likewise, if you initially inputted two milliliters of plasma into this process, your lower stop threshold range would be 51416960 EVs for analyzing the B cell EV population.

TABLE 4
Serial Capture Release Stop Thresholds for Analyzing
Tissue-Derived EVs. Estimated EV Threshold per mL
of Plasma Processed for Tissue-Derived EV Types.

| | Lower Range | Upper Range |
|---|---|---|
| Tissue-Derived EV Type | (EVs/mL) | (EVs/mL) |
| Adipose Tissue | 1637600 | 32752000 |
| Muscle | 122000 | 2440000 |
| Lung | 59200 | 1184000 |
| Liver | 36200 | 724000 |
| Nerve | 31400 | 628000 |
| Skin | 24600 | 492000 |
| Colon | 22600 | 452000 |
| Bladder | 17400 | 348000 |
| Kidney | 14600 | 292000 |
| Brain | 12800 | 256000 |
| Small Intestine | 6000 | 120000 |
| Esophagus | 5000 | 100000 |
| Pituitary | 5000 | 100000 |
| Heart | 4400 | 88000 |
| Pancreas | 800 | 16000 |
| Stomach | 400 | 8000 |

This table shows the stop thresholds for analyzing EVs in assay for cancers related to these tissue or organ-based EV types. Table 4 thresholds roughly encompass 10% to 200% of the average human's total EV concentration per specified organ or tissue type in plasma. Stopping serial capture release before this range (above 200%) likely increases the risk of generating false positives due to the abundance of nontarget EVs that likely will end up in the capillary analysis phase. Stopping serial capture release below the lower threshold may increase the risk of not detecting enough cancer correlates in the capillary analysis phase. That lack of signal detection tends to lead to an increased risk of false negatives. Like Table 3, Table 4 is for the volume of plasma initially inputted into the process. For example, if you initially inputted one milliliter of plasma into this process, your lower stop threshold range would be 59,200 EVs for analyzing the lung EV population.

TABLE 5
Serial Capture Release Stop Threshold for Analyzing
Sputum-Derived EVs. Estimated EV Threshold Range
per mL of Sputum for Lung Cancer.

| | Lower Range | Upper Range |
|---|---|---|
| Tissue-Derived EV Type | (EVs/mL) | (EVs/mL) |
| Lung | 25000 | 5000000 |

This table shows the stop thresholds for analyzing EVs for cancers related to these sputum-based EV types.

### Type 3: Number of EVs to Analyze Within Capillaries Once the Stop Threshold is Met.

TABLE 6
Resource Efficient Sample Sizes Based on Estimated
EV Count. Post-Threshold Met Ideal Sampling
Size for Single EV Capillary Analysis.

| Estimated EV Number within Threshold | Sample Size of EVs |
|---|---|
| 200 | 198 |
| 400 | 391 |
| 800 | 763 |
| 4,400 | 3478 |
| 5,000 | 3842 |
| 6,000 | 4406 |
| 12,800 | 7225 |
| 14,600 | 7765 |
| 17,400 | 8492 |
| 22,600 | 9566 |
| 24,600 | 9907 |
| 31,400 | 10854 |
| 36,200 | 11375 |
| 59,200 | 12957 |
| 88,000 | 13957 |
| 100,000 | 14227 |
| 120,000 | 14573 |
| 256,000 | 15578 |
| 292,000 | 15696 |
| 452,000 | 16000 |
| 492,000 | 16046 |
| 628,000 | 16160 |
| 724,000 | 16216 |
| 1,184,000 | 16358 |
| 32,752,000 | 16579 |
| 60,000,000 | 16583 |
| 79,440,800 | 16584 |
| 228,342,400 | 16586 |
| 1,017,960,000 | 16587 |

This table shows examples of ideal sampling ranges for various EV populations to go into the capillary phase. The values listed in this table ensures that you are approximately 99% confident that your sample size-going into the capillaries-represents the total population of EVs in the specified threshold range.

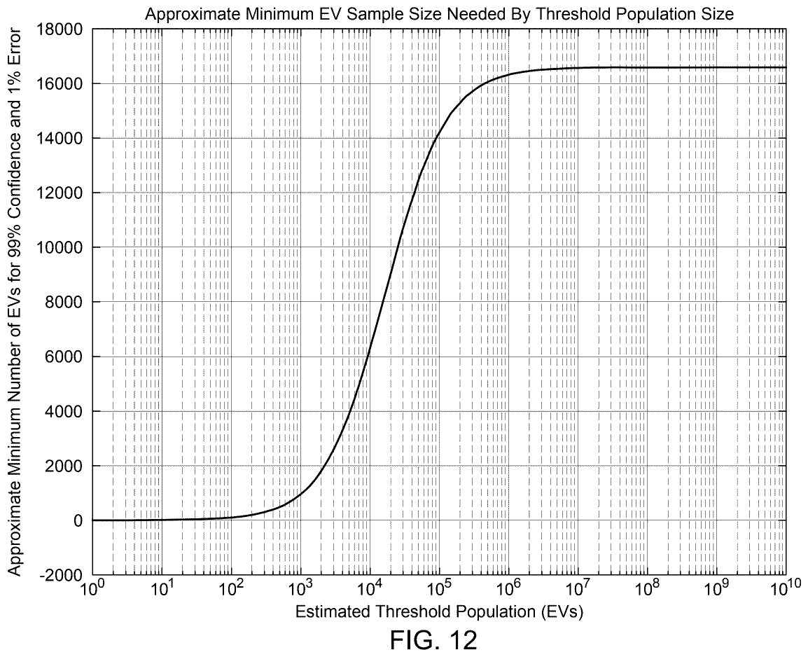

FIG. 12 shows Resource-efficient and statistically sound sample sizes based on threshold population size. This Figure is paired with Table 4 Data in that it fills gaps within the table.

### Numbering Scheme Legend:

1. Figure Methods Flow Chart;
2. Picture-Based Overview of Flow Showing Key Steps;
3. Figure Alternative Methods Flow Chart;
4. Sample that goes into assay;
5. Collect SEC Eluate;
6. EV-enriched eluate particle quantification;
7. Data store of total initial particles estimate;
8. Binder bound to beads to capture biomarkers;
9. Wash solution containing bound binders;
10. Binder-bead detachment;
11. Removes beads from solution;
12. EV quantification;
13. Estimated particle data storage after binder-bead detachment;
14. Decision and pathway if EVs fall within the desired range;
16. Decision and pathway if EVs fall below the desired range;
18. Process for increasing the amount of biofluid that gets processed for additional biomarker analysis;
20. Process for initially analyzing specific biomarkers;
21. Data store for the end of the serial capture release process;
22. Midprocess scoring algorithm;
24. Scoring algorithm decision junction;
26. Yes pathway;
27. Precapillary preparatory washing;
28. Precapillary binder detachment from beads;
29. Precapillary bead removal from solution;
30. Precapillary particle quantification;
31. Precapillary particle quantification data storage;
32. Process for diluting EVs to fall within desired EV quantification range;
34. Process for lysing EVs and keeping them inside the capillary bound to a wall;
35. Process for washing the unbound EV cargo and debris from the capillaries;
36. Denature the proteins inside the capillaries;
38. Process for analyzing denatured proteins and other biomolecules and then reading out the signal;
40. Process of aggregating all the collected data into the final scoring algorithm or equation and outputting a cancer result;
42. End process and keep cancer prediction;
44. Sample that goes into assay;
46. Perform SEC and collect SEC Eluate containing EV-enriched solution;
47. After EV-enriched eluate is collected, perform EV quantification, Data store of total initial particles estimate;
48. Proceed with serial capture and release of EVs until the threshold condition is met;
49. Zoom in of binder and binder fragments bound to EV which is also bound to a binder attached to a bead;
50. Probe emitting detectable signal;
51. Binder-bead detachment;
52. Fragment of a binder left on an EV from a past serial capture release cycle;
53. Section of bead;
54. Fragment of a binder left on EV from a past serial capture release cycle
56. Midassay data collecting;
58. Obtaining approximately one EV, lysing the Ev, and denaturing the protein steps, and adding binders and probes;
59. Show a fraction of the end of the second phase (lysed EV and amino acid sequence analysis portion);
60. A fraction of a denatured protein in its primary amino acid sequence;
62. Cancer-correlative portion of denatured protein;
64. Binder conjugated to probe emitting detectable signal;
66. The amalgamation of all the detectable signals get processed and analyzed;
108. Capillary containing approximately one EV in fluid;
110. Isotonic Solution;
112. Transmembrane Protein;
114. EV-to-binder-Capillary Connection;
116. EV;
118. Inside of EV Typically Containing a Subset of Cytosolic Cargo and fluid;
120. Interior of capillary walls containing lysing reagent resistant binders and blockers;
122. Majority of Isotonic Solution dispensed from capillary;
124. Capillary containing residual isotonic solution on its walls and approximately one EV;
126. Residual Fluid remains on the inner wall(s) of the capillary;
128. Lysing Solution Gets added to the Capillary;
129. Capillary Containing Lysing Solution and The resultant of Approximately One Lysed EV;
130. Solution Containing Lysing Solution;
134. Lysed EV debris (Partial EV membrane);
136. Transmembrane protein now bound to capillary wall binder on binding site that could not initially be access;
138. EV-to-binder-Capillary connection now largely removed from the remainder of the EV;
140. Unbound biomolecule released from interior of the EV;
142. Carbohydrate (which was initially inside the EV) biomarker bounder to binder on the capillary wall;
144. Wash away unbound particles, aspirate secondary binders which are conjugated to probes, wash away unbound secondary binders;
146. Capillary containing solution, bound biomarkers which also may have secondary binders conjugated to probes, and isotonic solution;
148. Capillary Solution;
150. Secondary binder (bound to transmembrane protein) conjugated to probe A;
152. Secondary binder (bound to 230) conjugated to probe B;
154. Secondary binder (bound to 234) conjugated to probe C;
156. A capillary that contains three EV-derived proteins (from approximately one EV);
158. Isotonic Solution;
160. An internal capillary wall coated with a variety of heat resistant, pH resistant, and lysing solution resistant binders and wall blockers;
162. Protein 1a bound to binder (which is covalently bound to the capillary wall);
164. Protein 2b bound to binder (which is covalently bound to the capillary wall);
166. Protein 3c bound to binder (which is covalently bound to the capillary wall);
168. Dispense Solution, aspirate binders conjugated to probes, and implement denaturation conditions (such as heating the capillary to 50° Celsius);
170. A capillary showing the resultant of 260's steps;
172. Unbound binder conjugated to Probe;
174. Binder (which is conjugated to a probe) bound to a cancer-correlative region on Protein 2b;
176. A partly unfolded Protein 2b;
178. Dispense solution, wash the capillary, and aspirate buffer;
180. A capillary showing the resultant of 270's steps;
182. Isotonic Solution;
184. Binder bound to Protein 2b;
186. Partly unfolded (but more folded) conformation of Protein 2b;

While this disclosure includes specific examples, it will be apparent after an understanding of the disclosure of this application has been attained that various changes in form and details may be made in these examples without departing from the spirit and scope of the claims and their equivalents.

1. A method of extracellular vesicle analysis for drastically reducing cross-contamination of intravesicular contents between neighboring extracellular vesicles comprising:
segregating extracellular vesicles from a solution such that approximately one of the extracellular vesicles enters each of a plurality of chambers;
lysing the segregated extracellular vesicles within said chambers to release intravesicular contents; and
analyzing said released intravesicular contents to detect disease-correlative biomarkers.

2. The method of claim 1, wherein the chambers comprise capillaries, and the segregating comprises aspirating the extracellular vesicles from a well-distributed pool of extracellular vesicles in the solution.

3. The method of claim 1, wherein the plurality of chambers comprises between 200 and 17,000 capillaries.

4. The method of claim 1, wherein the chambers comprise microfluidic compartments.

5. The method of claim 1, further comprising denaturing proteins within the released intravesicular contents to expose amino acid sequences correlative to disease.

6. The method of claim 5, wherein the denaturing comprises applying gradient-based denaturation to reveal secondary and primary structures of the proteins.

7. The method of claim 1, further comprising using mid-cycle quantification of the extracellular vesicles as a determinant for progressing to the segregating step, wherein the progressing occurs upon achieving a predetermined tissue-specific range.

8. The method of claim 7, wherein the predetermined tissue-specific range for lung tissue-derived extracellular vesicles in plasma comprises 59,200 to 1,184,000 EVs per mL of plasma processed.

9. The method of claim 1, wherein the lysing exposes biomarkers that are undetectable or less detectable when the extracellular vesicles are intact due to lipid membrane-based steric hindrance.

10. A method of detecting disease correlates using extracellular vesicles, whereby detection of the disease correlates occurs over a surface of the extracellular vesicles without proximity constraints, comprising:
obtaining a sample of biofluid;
using one or more binders to attach to extracellular vesicles correlative to a disease or target organ tissue from the biofluid;
measuring biomarkers on the attached extracellular vesicles;
upon the measurement not meeting a predetermined detection threshold, disconnecting the binders from the extracellular vesicles;
using one or more different binders to attach to the extracellular vesicles; and
repeating the disconnecting and the using one or more different binders until the measurement meets the predetermined detection threshold.

11. The method of claim 10, wherein concentrations of particles in the sample are measured.

12. The method of claim 11, wherein the concentrations are normalized to predetermined levels, whereby disease detection algorithms are optimized to account for factors influencing concentration variability.

13. The method of claim 10, wherein lung cancer is detected based on risk factors comprising one or more of smoking history, geography, genetics, age, symptoms, or diagnostic imaging results.

14. The method of claim 10, further comprising, after said predetermined detection threshold is met:
segregating said extracellular vesicles such that approximately one of the extracellular vesicles enters each of a plurality of chambers;
lysing the segregated extracellular vesicles within the chambers to release intravesicular contents; and
analyzing the released intravesicular contents,
whereby cross-contamination between the extracellular vesicles is drastically reduced during the analyzing.

15. The method of claim 14, wherein protein denaturing is performed on the released intravesicular contents under conditions sufficient to disrupt secondary structure.

16. The method of claim 10, wherein the binders are attached to ferromagnetic beads, and the disconnecting comprises detaching the binders from the beads using an enzyme or chemical reagent, and further comprising removing the beads from the solution using magnetic separation.

17. A method for detecting lung disease, whereby disease on interior lung regions exposed to sputum and disease on exterior lung regions exposed to blood are both detected for increased sensitivity, comprising:
obtaining a sample of sputum from a subject;
obtaining a sample of blood from the subject;
performing extracellular vesicle analysis on each of the samples; and
using results from both said samples to detect lung disease.

18. The method of claim 17, wherein the extracellular vesicle analysis comprises:
segregating extracellular vesicles such that approximately one of the extracellular vesicles enters each of a plurality of chambers;
lysing the segregated extracellular vesicles within the chambers to release intravesicular contents; and
analyzing the released intravesicular contents.

19. The method of claim 17, wherein the lung disease comprises early stage lung cancer.

20. The method of claim 17, wherein:
the extracellular vesicle analysis on the sample of blood uses a threshold range of 59,200 to 1,184,000 lung tissue-derived extracellular vesicles per mL of plasma processed; and
the extracellular vesicle analysis on the sample of sputum uses a threshold range of 25,000 to 5,000,000 lung tissue-derived extracellular vesicles per ml of sputum.

21. The method of claim 17, wherein the results from both samples are combined in a scoring algorithm to determine likelihood of lung disease.

22. The method of claim 17, further comprising denaturing proteins within the released intravesicular contents to expose amino acid sequences correlative to lung cancer.

23. A method of detecting a cancer-correlative biomarker in a biofluid sample from a subject, whereby segregation ensures approximate single-EV analysis to prevent cross-contamination, comprising:
accessing a biological sample obtained from the subject comprising extracellular vesicles;
segregating the extracellular vesicles such that approximately one of the extracellular vesicles enters each of a plurality of chambers;
lysing the segregated extracellular vesicles within the chambers to release intravesicular biomolecules, wherein the lysing renders a cancer-correlative biomarker detectable that is otherwise undetectable or less detectable in a non-lysed sample; and
detecting the released cancer-correlative biomarker.

24. The method of claim 23, wherein the detecting comprises correlating the detected biomarker with the presence, absence, likelihood, stage, subtype, or progression of cancer.

25. The method of claim 23, wherein the biomarker comprises one or more of amino acids, proteins, carbohydrates, or nucleic acids.

26. The method of claim 23, further comprising denaturing proteins within the released intravesicular biomolecules to expose amino acid sequences correlative to cancer.

27. A method of detecting a cancer-correlative biomarker in a biological sample from a subject, comprising:
providing a biological sample comprising extracellular vesicles obtained from said subject;
segregating the extracellular vesicles such that approximately one of the extracellular vesicles enters each of a plurality of chambers;
lysing the segregated extracellular vesicles within the chambers to release proteins;
partially denaturing the released proteins to expose secondary structural features while maintaining detectable primary sequence information; and
analyzing the exposed secondary structural features to identify cancer-correlative motifs.

28. The method of claim 27, further comprising further denaturing the proteins to expose primary structure and analyzing the primary structure for cancer-correlative amino acid sequences.

29. The method of claim 27, wherein the partially denaturing comprises applying a gradient of denaturing conditions to progressively expose structural features.

30. A method of approximate single extracellular vesicle analysis on a plurality of extracellular vesicles, whereby extracellular vesicles can be analyzed with reduced risk of cross-contamination from neighboring extracellular vesicles, and whereby steric hindrance from lipid membranes is reduced, comprising:
distributing the extracellular vesicles such that approximately one of the extracellular vesicles enters each of a plurality of chambers; and
lysing the distributed extracellular vesicles within the chambers to release intravesicular contents.

31. The method of claim 30, wherein proteins in the released intravesicular contents are exposed to conditions sufficient to destabilize hydrogen bonds and disulfide bonds for at least partial denaturation.

32. The method of claim 30, wherein binders specific to cancer-correlative amino acid sequences are added after the lysing.

33. The method of claim 30, wherein the extracellular vesicles are pre-filtered for a specific tissue type prior to the distributing.

34. The method of claim 33, wherein the specific tissue type is pulmonary.

35. The method of claim 30, wherein the chambers comprise capillaries pre-coated with a binding agent.

36. A system for patient-adaptive analysis of extracellular vesicles for disease detection, comprising:
supports configured to attach binders for serial capture and release of the extracellular vesicles; a detacher configured to disconnect the binders from the supports; a quantifier configured for mid-cycle quantification of the extracellular vesicles to determine progression based on a predetermined range; a plurality of chambers configured to segregate the extracellular vesicles such that approximately one of the extracellular vesicles enters each of the chambers; lysing reagents configured to lyse the segregated extracellular vesicles within the chambers to release intravesicular contents; and denaturation reagents configured for gradient-based denaturation of proteins within the released intravesicular contents.

37. The system of claim 36, wherein the supports comprise ferromagnetic beads, and the detacher comprises an enzyme or chemical reagent.

38. The system of claim 36, wherein the quantifier comprises a nanoparticle tracking analysis device or a resistive pulse sensing device, and is configured to normalize quantification for patient variability.

39. The system of claim 36, wherein the chambers comprise capillaries pre-coated with a binding agent, and further comprising a processor configured to apply a scoring algorithm to quantified biomarkers for determining disease likelihood.

40. The system of claim 36, further comprising an enrichment module configured to enrich the extracellular vesicles from a biofluid sample.

41. A system for performing extracellular vesicle-based disease detection, comprising:
binders on supports for serial capture and release of extracellular vesicles;
a detacher for disconnecting the binders from the supports;
a quantifier for mid-cycle extracellular vesicle counting;
a plurality of chambers for segregating the extracellular vesicles such that approximately one of the extracellular vesicles enters each of said chambers;
lysing reagents for lysing the segregated extracellular vesicles within the chambers to release intravesicular contents; and
denaturation reagents for denaturing proteins within said released intravesicular contents.

42. The system of claim 41, further comprising a processor for scoring based on quantified biomarkers from the released intravesicular contents.

43. The system of claim 41, wherein the chambers comprise capillaries configured to receive approximately one extracellular vesicle each from a well-distributed solution.
